SEQUENCE LISTING (15 sequences)

SEQ ID NO: 1 - DNA, 19 bp, synthetic construct
  source 1..19: mol_type = other DNA
taatacgactcactatagg

SEQ ID NO: 2 - DNA, 575 bp, synthetic construct
  source 1..575: mol_type = other DNA
cccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcaacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataat

SEQ ID NO: 3 - DNA, 678 bp, synthetic construct
  source 1..678: mol_type = other DNA
atggatagcactgagaacgtcatcaagcccttcatgcgcttcaaggtgcacatggagggctccgtgaacggccacgagttcgagatcgagggcgagggcgagggcaagccctacgagggcacccagaccgccaagctgcaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggtgaccgtgacccaggactcctccctgcaggacggcaccttcatctaccacgtgaagttcatcggcgtgaacttcccctccgacggccccgtaatgcagaagaagactctgggctgggagccctccaccgagcgcctgtacccccgcgacggcgtgctgaagggcgagatccacaaggcgctgaagctgaagggcggcggccactacctggtggagttcaagtcaatctacatggccaagaagcccgtgaagctgcccggctactactacgtggactccaagctggacatcacctcccacaacgaggactacaccgtggtggagcagtacgagcgcgccgaggcccgccaccacctgttccagtag

SEQ ID NO: 4 - DNA, 10 bp, synthetic construct
  source 1..10: mol_type = other DNA
gccaccatgg

SEQ ID NO: 5 - DNA, 1083 bp, synthetic construct
  source 1..1083: mol_type = other DNA
atggcgggacacctggcttcggatttcgccttctcgccccctccaggtggtggaggtgatgggccaggggggccggagccgggctgggttgatcctcggacctggctaagcttccaaggccctcctggagggccaggaatcgggccgggggttgggccaggctctgaggtgtgggggattcccccatgccccccgccgtatgagttctgtggggggatggcgtactgtgggccccaggttggagtggggctagtgccccaaggcggcttggagacctctcagcctgagggcgaagcaggagtcggggtggagagcaactccgatggggcctccccggagccctgcaccgtcacccctggtgccgtgaagctggagaaggagaagctggagcaaaacccggaggagtcccaggacatcaaagctctgcagaaagaactcgagcaatttgccaagctcctgaagcagaagaggatcaccctgggatatacacaggccgatgtggggctcaccctgggggttctatttgggaaggtattcagccaaacgaccatctgccgctttgaggctctgcagcttagcttcaagaacatgtgtaagctgcggcccttgctgcagaagtgggtggaggaagctgacaacaatgaaaatcttcaggagatatgcaaagcagaaaccctcgtgcaggcccgaaagagaaagcgaaccagtatcgagaaccgagtgagaggcaacctggagaatttgttcctgcagtgcccgaaacccacactgcagcagatcagccacatcgcccagcagcttgggctcgagaaggatgtggtccgagtgtggttctgtaaccggcgccagaagggcaagcgatcaagcagcgactatgcacaacgagaggattttgaggctgctgggtctcctttctcagggggaccagtgtcctttcctctggccccagggccccattttggtaccccaggctatgggagccctcacttcactgcactgtactcctcggtccctttccctgagggggaagcctttccccctgtctctgtcaccactctgggctctcccatgcattcaaactga

SEQ ID NO: 6 - DNA, 672 bp, synthetic construct
  source 1..672: mol_type = other DNA
atgccgcgcgtcgtgcccgaccagagaagcaagttcgagaacgaggagttttttaggaagctgagccgcgagtgtgagattaagtacacgggcttcagggaccggccccacgaggaacgccaggcacgcttccagaacgcctgccgcgacggccgctcggaaatcgcttttgtggccacaggaaccaatctgtctctccagttttttccggccagctggcagggagaacagcgacaaacacctagccgagagtatgtcgacttagaaagagaagcaggcaaggtatatttgaaggctcccatgattctgaatggagtctgtgttatctggaaaggctggattgatctccaaagactggatggtatgggctgtctggagtttgatgaggagcgagcccagcaggaggatgcattagcacaacaggcctttgaagaggctcggagaaggacacgcgaatttgaagatagagacaggtctcatcgggaggaaatggaggtgagagtttcacagctgctggcagtaactggcaagaagacaacaagacccatcagttctggaccagcgagctgtgctgcgactcgtggcgtaatcatggtcatagctgtttcctgtgtgaaattgttatccgctcacaattccacacaacatacgagccggaagcataaagtgtaa

SEQ ID NO: 7 - RNA, 1151 nt, synthetic construct
  source 1..1151: mol_type = other RNA
taatacgactcactatagggaaataagagagaaaagaagagtaagaagaaatataagagccaccatggatggcgggacacctggcttcggatttcgccttctcgccccctccaggtggtggaggtgatgggccaggggggccggagccgggctgggttgatcctcggacctggctaagcttccaaggccctcctggagggccaggaatcgggccgggggttgggccaggctctgaggtgtgggggattcccccatgccccccgccgtatgagttctgtggggggatggcgtactgtgggccccaggttggagtggggctagtgccccaaggcggcttggagacctctcagcctgagggcgaagcaggagtcggggtggagagcaactccgatggggcctccccggagccctgcaccgtcacccctggtgccgtgaagctggagaaggagaagctggagcaaaacccggaggagtcccaggacatcaaagctctgcagaaagaactcgagcaatttgccaagctcctgaagcagaagaggatcaccctgggatatacacaggccgatgtggggctcaccctgggggttctatttgggaaggtattcagccaaacgaccatctgccgctttgaggctctgcagcttagcttcaagaacatgtgtaagctgcggcccttgctgcagaagtgggtggaggaagctgacaacaatgaaaatcttcaggagatatgcaaagcagaaaccctcgtgcaggcccgaaagagaaagcgaaccagtatcgagaaccgagtgagaggcaacctggagaatttgttcctgcagtgcccgaaacccacactgcagcagatcagccacatcgcccagcagcttgggctcgagaaggatgtggtccgagtgtggttctgtaaccggcgccagaagggcaagcgatcaagcagcgactatgcacaacgagaggattttgaggctgctgggtctcctttctcagggggaccagtgtcctttcctctggccccagggccccattttggtaccccaggctatgggagccctcacttcactgcactgtactcctcggtccctttccctgagggggaagcctttccccctgtctctgtcaccactctgggctctcccatgcattcaaactga

SEQ ID NO: 8 - RNA, 701 nt, synthetic construct
  source 1..701: mol_type = other RNA
taatacgactcactatagggccaccatggatgccgcgcgtcgtgcccgaccagagaagcaagttcgagaacgaggagttttttaggaagctgagccgcgagtgtgagattaagtacacgggcttcagggaccggccccacgaggaacgccaggcacgcttccagaacgcctgccgcgacggccgctcggaaatcgcttttgtggccacaggaaccaatctgtctctccagttttttccggccagctggcagggagaacagcgacaaacacctagccgagagtatgtcgacttagaaagagaagcaggcaaggtatatttgaaggctcccatgattctgaatggagtctgtgttatctggaaaggctggattgatctccaaagactggatggtatgggctgtctggagtttgatgaggagcgagcccagcaggaggatgcattagcacaacaggcctttgaagaggctcggagaaggacacgcgaatttgaagatagagacaggtctcatcgggaggaaatggaggtgagagtttcacagctgctggcagtaactggcaagaagacaacaagacccatcagttctggaccagcgagctgtgctgcgactcgtggcgtaatcatggtcatagctgtttcctgtgtgaaattgttatccgctcacaattccacacaacatacgagccggaagcataaagtgtaa

SEQ ID NO: 9 - DNA, 1332 bp, synthetic construct
  source 1..1332: mol_type = other DNA
atggctacggccgcgtcaaaccactacagccttctgactagtagtgcctctattgttcacgcagagccaccgggtggaatgcaacaaggtgcggggggataccgcgaagcccagtcactggtacaaggagactatggggctttgcaatccaacggtcatcccctcagccatgcccaccaatggattaccgctctgagccatggcggaggaggtggaggtggagggggaggcggaggtggcggaggtgggggaggtggaggtggagatggaagcccctggagcacgtcccctcttggtcaaccggacatcaagcctagtgtagtcgtccagcagggcggccggggggatgaacttcatggacccggagcccttcagcaacaacatcaacagcaacagcaacaacagcagcaacagcagcaacaacaacaacaacagcaacagcagcaaaggcctccacacttggttcaccatgctgccaatcaccacccaggcccaggcgcatggaggtctgcggcagcggccgctcatttgccaccttccatgggagcttctaacggcggtcttctgtattcccagccctccttcacggtaaatggtatgctcggtgccggcggccaaccggccggactgcatcaccacgggcttcgagatgcccatgatgagccacaccacgctgatcaccacccacacccacattcacacccgcatcagcagcctcctcctccgccacccccgcaaggccctcctggacacccgggtgcccaccatgatccacattccgacgaggacacgcctacctctgatgatctggaacagtttgcaaagcagttcaaacaaaggcgaataaagctcggctttacgcaggcggacgtggggctcgcgctgggcacattgtatgggaacgtgttttcccaaacgaccatctgtcgctttgaagcgttgcaactcagcttcaaaaatatgtgcaagttgaaaccgcttcttaataagtggttggaggaagcggattccagttcaggctcaccaactagcattgacaaaatagctgcacaaggacgcaaaaggaagaagagaacatcaatagaggtttctgtaaagggggccttggaaagccacttcttgaagtgtcctaaaccctcagctcaggaaataacatctcttgctgattctttgcagttggaaaaggaggtagtgagggtctggttttgcaatcggcgccagaaagaaaagagaatgacgccacctggtgggacgctgcctggcgccgaggacgtctatgggggatcccgcgacacaccaccgcaccatggtgtgcagactccagtacaatga

SEQ ID NO: 10 - DNA, 92 bp, synthetic construct
  source 1..92: mol_type = other DNA
atagtgagtcgtattaaagcgtgaagatcctgttcaatattgctgtagtacaggtcgacgcaagcgtgaagatcctgttcaatattatccct

SEQ ID NO: 11 - DNA, 92 bp, synthetic construct
  source 1..92: mol_type = other DNA
atagtgagtcgtattaaatattgaacaggatcttcacgcttctaggctggacacacctctaaatattgaacaggatcttcacgcttatccct

SEQ ID NO: 12 - DNA, 22 bp, synthetic construct
  source 1..22: mol_type = other DNA
taatacgactcactatagggat

SEQ ID NO: 13 - RNA, 1361 nt, synthetic construct
  source 1..1361: mol_type = other RNA
taatacgactcactatagggccaccatggatggctacggccgcgtcaaaccactacagccttctgactagtagtgcctctattgttcacgcagagccaccgggtggaatgcaacaaggtgcggggggataccgcgaagcccagtcactggtacaaggagactatggggctttgcaatccaacggtcatcccctcagccatgcccaccaatggattaccgctctgagccatggcggaggaggtggaggtggagggggaggcggaggtggcggaggtgggggaggtggaggtggagatggaagcccctggagcacgtcccctcttggtcaaccggacatcaagcctagtgtagtcgtccagcagggcggccggggggatgaacttcatggacccggagcccttcagcaacaacatcaacagcaacagcaacaacagcagcaacagcagcaacaacaacaacaacagcaacagcagcaaaggcctccacacttggttcaccatgctgccaatcaccacccaggcccaggcgcatggaggtctgcggcagcggccgctcatttgccaccttccatgggagcttctaacggcggtcttctgtattcccagccctccttcacggtaaatggtatgctcggtgccggcggccaaccggccggactgcatcaccacgggcttcgagatgcccatgatgagccacaccacgctgatcaccacccacacccacattcacacccgcatcagcagcctcctcctccgccacccccgcaaggccctcctggacacccgggtgcccaccatgatccacattccgacgaggacacgcctacctctgatgatctggaacagtttgcaaagcagttcaaacaaaggcgaataaagctcggctttacgcaggcggacgtggggctcgcgctgggcacattgtatgggaacgtgttttcccaaacgaccatctgtcgctttgaagcgttgcaactcagcttcaaaaatatgtgcaagttgaaaccgcttcttaataagtggttggaggaagcggattccagttcaggctcaccaactagcattgacaaaatagctgcacaaggacgcaaaaggaagaagagaacatcaatagaggtttctgtaaagggggccttggaaagccacttcttgaagtgtcctaaaccctcagctcaggaaataacatctcttgctgattctttgcagttggaaaaggaggtagtgagggtctggttttgcaatcggcgccagaaagaaaagagaatgacgccacctggtgggacgctgcctggcgccgaggacgtctatgggggatcccgcgacacaccaccgcaccatggtgtgcagactccagtacaatga

SEQ ID NO: 14 - RNA, 92 nt, synthetic construct
  source 1..92: mol_type = other RNA
agggataatattgaacaggatcttcacgcttgcgtcgacctgtactacagcaatattgaacaggatcttcacgctttaatacgactcactat

SEQ ID NO: 15 - RNA, 92 nt, synthetic construct
  source 1..92: mol_type = other RNA
agggataagcgtgaagatcctgttcaatatttagaggtgtgtccagcctagaagcgtgaagatcctgttcaatatttaatacgactcactat

(19) United States
(12) Patent Application Publication
(10) Pub. No.: US 2025/0002857 A1
(43) Pub. Date: Jan. 2, 2025

(54) Composition for inducing direct reprogramming comprising self-assembled RNA construct

(71) Applicant: UNIVERSITY OF SEOUL INDUSTRY COOPERATION FOUNDATION, Seoul (KR)

(72) Inventors: Jong Bum Lee, Seoul (KR); Hyejin Kim, Seoul (KR)

(21) Appl. No.: 18/700,687

(22) PCT Filed: Oct. 20, 2022
(86) PCT No.: PCT/KR2022/015999

§ 371 Date: Apr. 11, 2024

(30) Foreign Application Priority Data
Oct. 20, 2021 (KR) 10-2021-0140465

(51) Int. Cl. C12N 5/077; C12N 5/079; C12N 15/113; C12N 15/90

(52) U.S. Cl. CPC C12N 5/0654; C12N 5/0618; C12N 15/113; C12N 15/90; C12N 2310/14; C12N 2501/603; C12N 2506/1307

(57) ABSTRACT

The composition for inducing direct reprogramming of the present disclosure, which is formed by self-assembly of transcripts comprising, as a repeating unit sequence, mRNA for the expression of a transcription factor, or RNA for RNA interference to inhibit the expression of a transcription factor can induce direct reprogramming in a stable and efficient manner.

### TECHNICAL FIELD

The present disclosure claims priority based on Korean Patent Application No. 10-2021-0140465 filed on Oct. 20, 2021.

The present disclosure relates to a composition for inducing direct reprogramming including a self-assembled RNA construct.

### BACKGROUND ART

Recently, the demand for cell therapy and disease models for various incurable diseases has been rapidly increasing. Accordingly, patient-specific cell therapy using induced pluripotent stem cells (iPSCs) has been studied. IPSCs refer to cells that have been dedifferentiated into a state of embryonic stem cells with pluripotency capable of being differentiated into all types of cells by injecting a dedifferentiation-inducing protein into a somatic cell. Despite the advantage of pluripotency, the IPSCs may remain in an undifferentiated state that is not sufficiently differentiated during the process of differentiating these cells into specific cells, and when these undifferentiated IPSCs are transplanted into the body, there is a risk of causing cancer and genetic modification, thereby limiting the application of the IPSCs to humans.

As one of the ways to solve the above problems, a direct reprogramming technology, which induces direct reprogramming to different types of cells by introducing a transcription factors into a somatic cell, is attracting attention. Direct reprogramming differs from the technology using IPSCs in that it does not undergo dedifferentiation or re-differentiation. The above direct reprogramming technology can solve the problems of induced pluripotent stem cells, but the protocol has not been standardized to such an extent that different researchers use a different transcription factor a gene, and the mechanism of action has not been clearly identified. In addition, there are many problems in the direct reprogramming in that there is a difficulty in discovering transcription factors for direct reprogramming, that the conversion efficiency of discovered transcription factors is often low, and that even if transcription factors are morphologically converted, they may do not show physiological functionality.

Additionally, in gene delivery for direct reprogramming, virus- or DNA-based delivery carries the risk of infection or permanent modification of a gene. There have been attempts to transfer a gene using RNA to exclude the above risks, but RNA has a problem in that it is unstable and has low conversion efficiency.

### PRIOR ART DOCUMENT

### Patent Document

Korean Patent Application Publication No. 10-2020-0004744

### DETAILED DESCRIPTION OF INVENTION

### Technical Problems

According to a specific embodiment of the present disclosure, there is provided a composition for inducing direct reprogramming based on a three-dimensional RNA construct, which, being based on RNA, has no risk of infection or genetic modification, and can induce direct reprogramming with stability and high efficiency.

### Technical Solution

Terms which are not specifically defined in this specification should be understood as having meanings commonly used in the technical field to which the present invention pertains. Additionally, unless specifically defined in context, a singular includes a plural, and a plural includes a singular.

In an aspect, the present disclosure provides a composition for inducing direct reprogramming, which includes: a first active ingredient comprising a three-dimensional RNA construct, in which one or two types of transcripts, each having a repeating unit sequence for the expression of a transcription factor, are mixed and self-assembled; a second active ingredient comprising a three-dimensional RNA construct, in which one or two types of transcripts, each having a repeating unit sequence for inhibiting the expression of a transcription factor, are mixed and self-assembled; or a combination thereof.

The “three-dimensional RNA construct” refers to a three-dimensional particle formed by self-assembly through a process of entanglement or aggregation of transcripts or a group of transcripts.

The “direct reprogramming” refers to inducing a conversion from a differentiated cell into a desired cell. The direct reprogramming differs from a conversion process using induced pluripotent stem cells (iPSCs), which requires a redifferentiation process, in that it does not undergo a redifferentiation process.

The “transcription factor” refers to a protein which binds to the response element of a gene and thereby promotes or inhibits expression of the gene.

The unit sequence for expression of the transcription factor may include the mRNA sequence of the transcription factor, a ribosome binding site, and a combination thereof which are required to induce direct reprogramming, for example, the one in which the promoter, the ribosome binding site, and the mRNA sequence of transcription factor are operably linked. The “operably linked” means that the expression control sequence and the gene sequence are arranged to function together, for example, promoting mRNA transcription of a transcription factor by allowing these sequences to be directly linked or arranged closely therebetween. The unit sequence for suppressing the expression of the transcription factor may be a sequence of an RNA molecule capable of inhibiting the expression of the desired transcription factor by RNA interference, for example, a sequence of a siRNA, shRNA, or miRNA.

The transcript refers to a long sequence in which a unit sequence is repeated, and the number of repetitions may be 1 to 1,000, 1 to 500, 1 to 300, 1 to 200, 1 to 100, 10 to 1,000, 10 to 500, 10 to 300, 10 to 200, or 10 to 100, but is not limited thereto.

According to an embodiment, the size (diameter) of the three-dimensional RNA construct may be 50 nm to 1,000 nm, 50 nm to 500 nm, 50 nm to 400 nm, 50 nm to 300 nm, 50 nm to 250 nm, 50 nm to 200 nm, 100 nm to 1,000 nm, 100 nm to 500 nm, 100 nm to 400 nm, 100 nm to 300 nm, 100 nm to 250 nm, or 100 nm to 200 nm.

According to a specific embodiment, the average size (diameter) of the three-dimensional RNA construct may be 100 nm to 800 nm, 100 nm to 700 nm, 100 nm to 600 nm, 100 nm to 500 nm, 100 nm to 400 nm, 100 nm to 300 nm, or 100 nm to 200 nm.

The size of the three-dimensional RNA construct is related to the length and concentration of the transcript and the copy number of the unit sequence included therein. When the size of the three-dimensional RNA construct is large, the efficiency of transfection into cells decreases, but due to the presence of a large number of unit sequences, the three-dimensional RNA construct can increase the efficiency of expression or inhibition of transcription factors, and can have a long duration by delaying its intracellular decomposition. In contrast, when the size of the three-dimensional RNA construct is small, the three-dimensional RNA construct can increase transfection efficiency; however, due to the presence of a small number of unit sequences, the three-dimensional RNA construct may reduce the efficiency of expression or inhibition of transcription factors and may easily be decomposed within cells, and thus may have a short duration. According to an embodiment, the efficiency of transfection and direct reprogramming can be high when the average size of the three-dimensional RNA construct is in the range of 100 nm to 400 nm.

According to an embodiment, the transcript may be transcribed by rolling circle transcription. Rolling circle transcription is a method of synthesizing a single long transcript with a repeating unit sequence by removing the transcription termination sequence of a plasmid, and is well known to those skilled in the art.

According to an embodiment, a three-dimensional RNA construct may be synthesized by supplying a sufficiently high concentration of ribonucleotide triphosphate (rNTP) (1 mM or higher for each of rATP, rUTP, rGTP, and rCTP), supplying the concentration of plasmid DNA at a concentration of 0.1 nM or higher, which is considered as the limiting concentration at which three-dimensionalization does not occur easily, and maintaining the reaction for a certain period of time (e.g., 12 hours or more) at the enzyme reaction temperature of 37° C. so that the reaction of forming the three-dimensional structure formation is saturated. Since it is difficult to specify the length of each RNA due to the nature of the rolling circle transcription (RCT) reaction, an appropriate time-point of termination for the reaction may be the time-point at which the size of the three-dimensional construct reaches the target value (e.g., an average diameter of 100 nm to 400 nm).

According to a specific embodiment, the three-dimensional RNA construct may maintain the expression activity of a transcription factor or the activity of inhibiting the expression of a transcription factor for 30 days or more after transfection into cells. In general, when mRNA, siRNA, shRNA, or miRNA is injected into cells to promote or inhibit the expression of a target gene, the duration is only a few hours, thus making it difficult to use the same as a gene delivery method. However, it was confirmed that in the injection of a three-dimensional RNA construct in the form of a self-assembled transcript having a long repeating unit sequence, the activity of promoting or inhibiting expression can be sustained for 30 days or more with just one injection, and direct reprogramming can successfully occur.

According to an embodiment, the composition for inducing direct reprogramming may induce direct reprogramming through one-time transfection into cells.

The three-dimensional RNA construct of the first active ingredient may be one in which transcripts of one type having a repeating unit sequence for the expression of a transcription factor are self-assembled, or one in which two types of transcripts expressing mutually different transcription factors are mixed and self-assembled. According to an embodiment, an RNA construct self-assembled by mixing a transcript for OCT4 expression and a transcript for CBF-β expression showed an increased efficiency of direct reprogramming compared to RNA constructs in which each of the transcripts was self-assembled.

The three-dimensional RNA construct of the second active ingredient may be one in which transcripts of one type having a repeating unit sequence for inhibiting the expression of a transcription factor are self-assembled, or one in which transcripts having a repeating unit sequence (a sense sequence) for inhibiting the expression and transcripts having a repeating unit sequence (an antisense sequence) that can complement the same are mixed and self-assembled. According to an embodiment, an RNA construct, in which a transcript having a repeating sense sequence and a transcript having a repeating antisense sequence are mixed and self-assembled so as to express siRNA (double-stranded) for inhibiting the expression of PTB, was shown to effectively inhibit the PTB expression.

According to a specific embodiment, the first active ingredient may include each of a three-dimensional RNA construct, in which the transcripts having a repeating unit sequence for the expression of octamer-binding transcription factor 4 (OCT4) are self-assembled, and a three-dimensional RNA construct, in which the transcript having a repeating unit sequence for the expression of core-binding factor subunit beta (CBF-β) are self-assembled; or a three-dimensional RNA construct, in which the transcripts having a repeating unit sequence for the expression of OCT4 and the transcripts having a repeating unit sequence for the expression of CBF-β are mixed and self-assembled; wherein the direct reprogramming is a direct reprogramming from a somatic cell to an osteoblast.

According to an embodiment, the three-dimensional RNA constructs expressing OCT4 and CBF-β successfully induced direct reprogramming from fibroblasts to osteoblasts, and were confirmed to exhibit not only morphological changes but also physiological functions.

The “octamer-binding transcription factor 4 (OCT4)” is also referred to as a POU domain, class 5, transcription factor 1 (POU5F1), and it refers to a protein encoded by the POU5F1 gene in humans, which is known to play an important role in self-renewal of undifferentiated embryonic stem cells.

The “core-binding factor subunit beta (CBF-β)” refers to a protein encoded by the CBF-β gene belonging to the PEBP2/CBF transcription factor family.

According to a specific embodiment, the unit sequence for OCT4 expression may consist of the sequence of SEQ ID NO: 7, and the unit sequence for CBF-β expression may consist of the sequence of SEQ ID NO: 8.

According to a specific embodiment, the first active ingredient may include a three-dimensional RNA construct, in which the transcripts having a repeating unit sequence for the expression of BRN2 are self-assembled; and the second active ingredient may include a three-dimensional RNA construct, in which the transcripts having a repeating unit sequence for inhibiting the expression of polypyrimidine tract binding protein (PTB) are self-assembled; wherein the direct reprogramming may be a direct reprogramming from a somatic cell to a nerve cell.

The “BRN2” is also referred to as POU3F2, and is known to be involved in the differentiation of nerve cells and the activity of corticotrophin releasing hormone.

The “polypyrimidine tract binding protein (PTB)” is also referred to as hnRNP I, and is known to play roles such as a splicing regulator, mRNA stability, and RNA localization.

According to an embodiment, the three-dimensional RNA construct expressing BRN2 and the three-dimensional RNA construct expressing siRNA, which inhibits the expression of PTB, successfully induced direct reprogramming from a fibroblast to a nerve cell, and were confirmed to exhibit not only morphological changes but also physiological functions.

According to a specific embodiment, the unit sequence for BRN2 expression may consist of the sequence of SEQ ID NO: 13; the unit sequence for PTB expression may consist of the sequence of SEQ ID NO: 14 or SEQ ID NO: 15; wherein the three-dimensional RNA construct of the second active ingredient may be one in which the transcripts having a repeating unit sequence of SEQ ID NO: 14, the transcripts having a repeating unit sequence of SEQ ID NO: 15, or mixed transcripts thereof are self-assembled.

According to a specific embodiment, the composition for inducing direct reprogramming may be for transfection into any one cell selected from the group consisting of fibroblasts, muscle cells, nerve cells, gastric mucosa cells, goblet cells, G cells, pericytes, astrocytes, B cells, blood cells, epithelial cells, neural stem cells, hematopoietic stem cells, mesenchymal stem cells, and umbilical cord blood stem cells.

Another aspect of the present disclosure provides a method for inducing direct reprogramming, which includes (a) delivering the composition for inducing direct reprogramming to somatic cells; and (b) culturing the somatic cells into which the three-dimensional RNA construct has been introduced by the delivery.

The medium used for the culture in step b) may include all media commonly used in the art including carbon sources, nitrogen sources, and trace element components, and may include all media commonly used for culturing stem cells, progenitor cells, etc., as well as somatic cell culture media.

### Effect of Invention

The composition for inducing direct reprogramming according to an embodiment can induce direct reprogramming of somatic cells into target cells of different lineages, and the cells converted through direct reprogramming retain physiological functionality.

The composition for inducing direct reprogramming according to an embodiment can exclude the risk of infection or permanent genetic modification of conventional virus- or DNA-based gene delivery methods by including RNA prepared in the form of a three-dimensional construct, and although the method is an RNA-based gene delivery method, it can have stability and high cell conversion efficiency.

### BRIEF DESCRIPTION OF THE DRAWING

FIG. 1 shows the analysis results of the size of a three-dimensional RNA construct using dynamic light scattering (DLS).

FIG. 2 shows the results of confirming the RNA constitution of a three-dimensional RNA construct using nanoparticle tracking analysis.

FIG. 3 shows the results of confirming whether or not the three-dimensional RNA construct was three-dimensionally assembled using an atomic force microscope.

FIG. 4 shows the results of confirming the stability of a three-dimensional RNA construct in an in vivo-like environment.

FIG. 5 shows the results of confirming the safety of a three-dimensional RNA construct.

FIG. 6 shows the results of delivering RNA using a three-dimensional RNA construct and tracking the expression of a target protein (a red fluorescent protein) for 15 days. Mean fluorescence intensity (MFI); average fluorescence intensity. Statistical analysis was performed using student's t-test (** p<0.01, *** p<0.001, **** p<0.0001).

FIG. 7 shows the results of confirming whether or not fibroblasts, after a three-dimensional RNA construct expressing OCT4 and CBF-β was delivered thereto, were directly reprogrammed into osteoblasts.

FIG. 8 shows the results of confirming the expression level of OPN, which is an osteoblast-specific gene, by measurement.

FIG. 9 shows the results of confirming calcium accumulation by Alizarin Red S (ARS) staining on the 15th and 21st days after treatment with a three-dimensional RNA construct.

FIG. 10 shows the results of ARS staining and quantitative analysis in a case where a three-dimensional RNA construct for OCT4 and a three-dimensional RNA construct for CBF-β were mixed and applied (individual) and a case where a single three-dimensional RNA construct simultaneously including two types of mRNA (i.e., OCT4 and CBF-β) (mixed).

FIG. 11 shows the results of fluorescence analysis of the production of calcium deposits due to osteoblast production through Osteo-Image mineralization analysis.

FIG. 12 shows the analysis results of the expression levels of OPN and Runx2 through quantitative RT-PCR.

FIG. 13 shows the results of confirming whether or not fibroblasts, after a three-dimensional RNA construct was delivered thereto, were converted into nerve cells through direct reprogramming.

FIG. 14 shows the analysis results of the expression levels of Tuj1 and MAP2, which are neuron-specific genes, in directly reprogrammed central neurons using the immunostaining technique.

FIG. 15 shows the results of confirming the expression levels of BRN2 and PTB using the immunostaining technique 30 days after treatment with a three-dimensional RNA construct.

### BEST MODE FOR CARRYING OUT THE INVENTION

Hereinafter, the present disclosure will be described in more detail by way of examples. However, these examples are for illustrative purposes only, and the scope of the present disclosure is not limited by these examples. Additionally, terms that are not specifically defined in this specification should be understood as having meanings commonly used in the art to which the present disclosure pertains.

### Example 1. Preparation of Three-Dimensional RNA Construct Expressing DsRed-Express2 and Confirmation of Effect Thereof

### 1-1. Preparation of Three-Dimensional RNA Construct Expressing DsRed-Express2

Integrated DNA Technologies, Inc. was contacted to prepare a plasmid (pIDTSmart(Amp) or pUCIDT(Amp) into which the DsRed-Express2 mRNA encoding sequence was inserted and the transcription termination site was removed. The DsRed-Express2 mRNA encoding sequence is operably linked to the T7 promoter and an internal ribosomal entry site (IRES).

The T7 promoter, IRES, and DsRed-Express2 mRNA sequence included in the plasmid are shown in SEQ ID NOS: 1 to 3 in Table 1 below. The plasmid may further include a replication origin and antibiotic resistance gene sequences, but these sequences are not related to the function of the three-dimensional RNA construct intended in the present disclosure, and thus they are omitted herein.

TABLE 1

| | SEQ | |
|---|---|---|
| Type | ID NO | Sequence |
| T7 | 1 | TAATACGACTCACTATAGG |
| promoter | | |
| IRES | 2 | CCCCTCTCCCTCCCCCCCCCCTAACGTTACTGGCCGAAGCCGCT |
| | | TGGAATAAGGCCGGTGTGCGTTTGTCTATATGTTATTTTCCACC |
| | | ATATTGCCGTCTTTTGGCAATGTGAGGGCCCGGAAACCTGGCC |
| | | CTGTCTTCTTGACGAGCATTCCTAGGGGTCTTTCCCCTCTCGCC |
| | | AAAGGAATGCAAGGTCTGTTGAATGTCGTGAAGGAAGCAGTT |
| | | CCTCTGGAAGCTTCTTGAAGACAAACAACGTCTGTAGCGACCC |
| | | TTTGCAGGCAGCGGAACCCCCCACCTGGCAACAGGTGCCTCTG |
| | | CGGCCAAAAGCCACGTGTATAAGATACACCTGCAAAGGCGGC |
| | | ACAACCCCAGTGCCACGTTGTGAGTTGGATAGTTGTGGAAAGA |
| | | GTCAAATGGCTCTCCTCAAGCGTATTCAACAAGGGGCTGAAGG |
| | | ATGCCCAGAAGGTACCCCATTGTATGGGATCTGATCTGGGGCC |
| | | TCGGTGCACATGCTTTACATGTGTTTAGTCGAGGTTAAAAAAA |
| | | CGTCTAGGCCCCCCGAACCACGGGGACGTGGTTTTCCTTTGAA |
| | | AAACACGATGATAAT |
| DsRed- | 3 | ATGGATAGCACTGAGAACGTCATCAAGCCCTTCATGCGCTTCA |
| Express2 | | AGGTGCACATGGAGGGCTCCGTGAACGGCCACGAGTTCGAGA |
| mRNA | | TCGAGGGCGAGGGCGAGGGCAAGCCCTACGAGGGCACCCAGA |
| | | CCGCCAAGCTGCAGGTGACCAAGGGCGGCCCCCTGCCCTTCGC |
| | | CTGGGACATCCTGTCCCCCCAGTTCCAGTACGGCTCCAAGGTG |
| | | TACGTGAAGCACCCCGCCGACATCCCCGACTACAAGAAGCTGT |
| | | CCTTCCCCGAGGGCTTCAAGTGGGAGCGCGTGATGAACTTCGA |
| | | GGACGGCGGCGTGGTGACCGTGACCCAGGACTCCTCCCTGCAG |
| | | GACGGCACCTTCATCTACCACGTGAAGTTCATCGGCGTGAACT |
| | | TCCCCTCCGACGGCCCCGTAATGCAGAAGAAGACTCTGGGCTG |
| | | GGAGCCCTCCACCGAGCGCCTGTACCCCCGCGACGGCGTGCTG |
| | | AAGGGCGAGATCCACAAGGCGCTGAAGCTGAAGGGCGGCGGC |
| | | CACTACCTGGTGGAGTTCAAGTCAATCTACATGGCCAAGAAGC |
| | | CCGTGAAGCTGCCCGGCTACTACTACGTGGACTCCAAGCTGGA |
| | | CATCACCTCCCACAACGAGGACTACACCGTGGTGGAGCAGTAC |
| | | GAGCGCGCCGAGGCCCGCCACCACCTGTTCCAGTAG |

A three-dimensional RNA construct, in which transcripts including a plurality of repeated DsRed-Express2 mRNA sequences are self-assembled, was prepared by transcribing the plasmid at high concentration using the rolling circle transcription (RCT) method. When the rolling circle transcription (RCT) reaction is performed under conditions where the concentration of ribonucleotide triphosphate (rNTP) and the concentration of the plasmid are above a certain level, long transcripts can self-assemble by tangling and twisting to thereby form a three-dimensional construct. For example, when the rNTP concentration for each of rATP, rUTP, rGTP, and rCTP is in the range of 1 mM to 8 mM, and the plasmid DNA concentration is in the range of 0.1 nM to 20 M, if the transcription reaction is performed at 37° C. for 12 hours or more, a three-dimensional RNA construct can be synthesized. No separate RNA enrichment procedure is required. Due to the nature of the RCT reaction, it is difficult to specify the length of each transcript. Therefore, the termination time-point of the RCT reaction is set at the time-point when the three-dimensional RNA construct reaches a certain size. For example, the reaction time can be set at the time-point when the average diameter of the three-dimensional RNA construct reaches the size of about 100 nm to 400 nm, 100 nm to 300 nm, or 100 nm to 200 nm.

The three-dimensional RNA construct prepared above was injected into cells to confirm whether red fluorescent protein (DsRed-Express2) was stably and continuously expressed in the cells. Specifically, 5 nM of plasmid DNA, a 4 mM ribonucleotide solution mix (New England Biolabs), a reaction buffer (80 mM Tris-HCl, 4 mM spermidine, 12 mM MgCl₂, and 2 mM dithiothreitol), and 50 units/mL of T7 RNA polymerase (New England Biolabs) were added to a tube and mixed, and then the mixture was reacted in an incubator at 37° C. for 20 hours, and thereby a three-dimensional RNA construct, in which transcripts including the DsRed-Express2 mRNA region are self-assembled, was synthesized.

### 1-2. Confirmation of Characteristics of Three-Dimensional RNA Construct Expressing DsRed-Express2

The size of the three-dimensional RNA construct expressing DsRed-Express2 prepared in Example 1-1 was analyzed using dynamic light scattering (DLS).

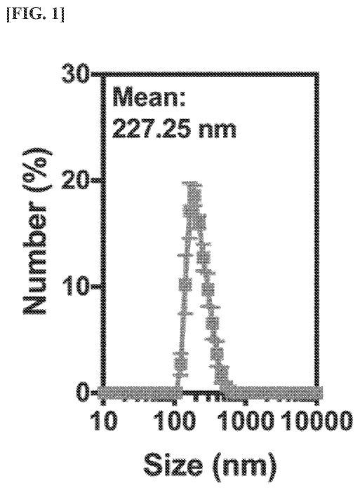

According to FIG. 1, it was confirmed that the three-dimensional RNA construct expressing DsRed-Express2 has a diameter of about 100 nm to about 1,000 nm (average 227.25 nm) in an aqueous solution.

It was possible to control the size of the three-dimensional RNA construct by changing the concentration of the reactants. The average size of the three-dimensional construct was increased as the concentration of the template plasmid DNA was increased from 1 nM to 25 nM, as the concentration of the ribonucleotide triphosphate mix as a reactant was increased from 4 mM to 8 mM, and as the concentration of T7 RNA polymerase as a reaction enzyme was lowered from 5,000 units/mL to 50 units/mL.

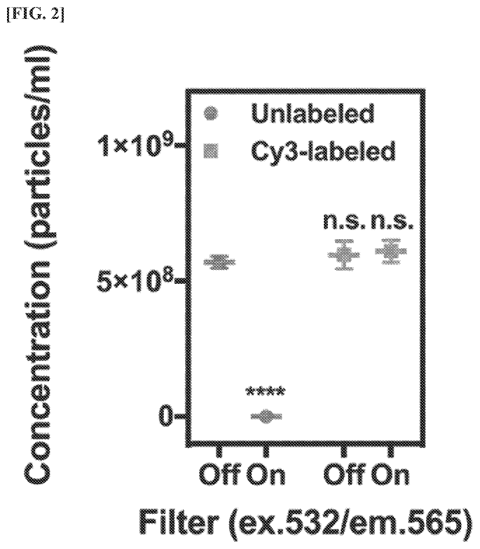

Additionally, in order to confirm whether the three-dimensional RNA construct expressing DsRed-Express2 includes the mRNA of DsRed-Express2, a three-dimensional RNA construct expressing DsRed-Express2 was prepared by transcribing RNA with fluorescently labeled UTP (cyanine 3-labeled UTP; cy3-labeled UTP), and fluorescent labeling of the final three-dimensional RNA construct was confirmed by the nanoparticle tracking analysis. According to FIG. 2, it was confirmed that the three-dimensional RNA construct expressing DsRed-Express2 includes DsRed-Express2 mRNA.

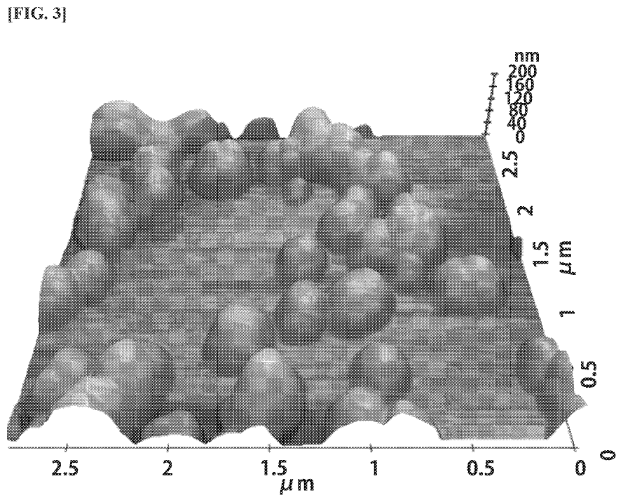

Additionally, the image of the three-dimensional RNA construct expressing DsRed-Express2 was reprogrammed in three dimensions using an atomic force microscope with excellent resolution in the height direction so as to confirm the presence of a three-dimensional assembly. As shown in FIG. 3, it was confirmed that the RNA construct expressing DsRed-Express2 was assembled in three dimensions.

### 1-3. Confirmation of Stability of Three-Dimensional RNA Construct

The stability of the three-dimensional RNA construct expressing DsRed-Express2 was evaluated in medium conditions containing nucleases.

Specifically, as the medium conditions, general human fibroblast culture conditions containing 10% fetal bovine serum (FBS) and antibiotics (antibiotic-antimycotic; final concentrations of 100 units/mL of penicillin, 100 μg/mL of streptomycin, and 0.25 μg/mL of Gibco Amphotericin B) to the Dulbecco's Modified Eagle's Medium (DMEM; high glucose) were used. The fetal bovine serum contained nucleases. The three-dimensional RNA construct expressing DsRed-Express2 prepared in Example 1-1 was dispensed into the medium at the same concentration and incubated at 37° C., which was the cell culture temperature, for 1, 6, 12, 24, and 60 hours, respectively. A three-dimensional RNA construct expressing DsRed-Express2 without any treatment (indicated as 0 time) was used as a control. The control and experimental groups were subjected to polyacrylamide gel electrophoresis (10% polyacrylamide gel, 100 V, 60 minutes), and the band sizes of residual RNA with high molecular weight were confirmed.

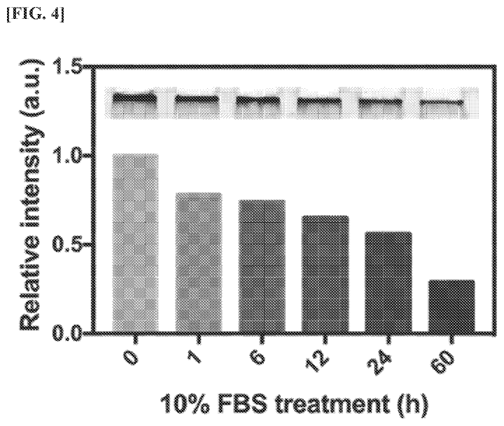

According to FIG. 4, the three-dimensional RNA construct expressing DsRed-Express2 showed a strong RNA band with a high molecular weight of several kilobases or more even after 60 hours in medium conditions containing nucleases. When DsRed-Express2 mRNA exists as a single strand without forming a three-dimensional construct, it is completely degraded within a few minutes under the same medium conditions, and in consideration of the same, the three-dimensional RNA construct expressing DsRed-Express2 has significantly higher resistance to nucleases and safety therefrom.

### 1-4. Confirmation of Safety of Three-Dimensional RNA Construct Expressing DsRed-Express2

A three-dimensional RNA construct expressing DsRed-Express2 was transfected into primary human dermal fibroblasts (HDFs) and the viability of the cells was evaluated.

Specifically, 5,000 human fibroblasts were dispensed per well into a 96-well cell culture plate and incubated for 24 hours. A three-dimensional RNA construct expressing DsRed-Express2 at a concentration of 0.1 μg, 0.5 μg, 2 μg, and 10 μg per 1 mL culture medium (a total medium volume of 100 μL) was mixed according to the manufacturer's protocol with a transfection reagent of TransIT-X2 dynamic delivery system (Mirus Bio LLC), respectively, and treated on the cells, and then incubated again for 24 hours. The resulting cells were treated with WST-8 reagent, a cytotoxicity evaluation reagent, and changes in cell viability were evaluated compared to the untreated control group (expressed as 0 μg/mL). Four wells each were tested under the same conditions, and the average values of each group were compared.

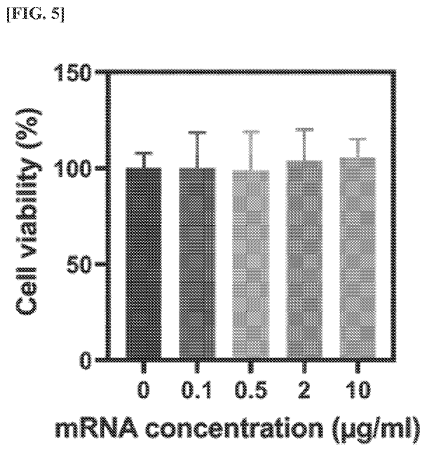

According to FIG. 5, the three-dimensional RNA construct expressing DsRed-Express2 showed excellent cell viability even when transfected into cells at a high concentration of 10 μg/mL, thus confirming its high level of safety.

### 1-5. Confirmation of Gene Expression Ability of Three-Dimensional RNA Construct Expressing DsRed-Express2

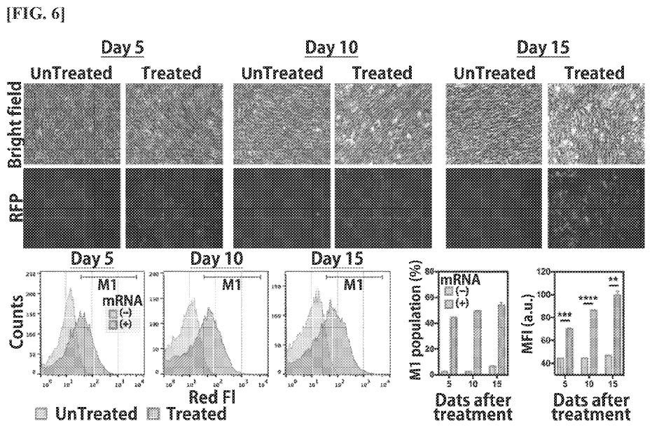

It was confirmed whether the three-dimensional RNA construct expressing DsRed-Express2 transfected into cells could express the DsRed-Express2 gene. A three-dimensional RNA construct expressing DsRed-Express2, which was synthesized with an average size of 100 nm to 200 nm, was prepared at a concentration of 4 μg/mL by adjusting the plasmid concentration based on the method of Example 1-1 above, and the resulting construct was mixed with the transfection reagent of the TransIT-X2 dynamic delivery system (Mirus Bio LLC) according to the manufacturer's protocol, and the mixture was treated once on human fibroblasts (indicated as treated in FIG. 6) and incubated.

On the 5th, 10th, and 15th day after the start of incubation, the expression level of red fluorescent protein was confirmed using fluorescence microscopic images and flow cytometry, and compared with the untreated control group (indicated as untreated in FIG. 6).

According to FIG. 6, in the cells to which the three-dimensional RNA construct expressing DsRed-Express2 was delivered, the number of red fluorescent protein-expressing cells (M1) and the mean fluorescence intensity (MFI) of the cells continued to increase for 15 days. This confirmed that genes can be expressed stably and efficiently merely by one-time transfection of the three-dimensional RNA construct into cells, and this result is contrary to the fact that several rounds of mRNA delivery are generally required for the expression of a target protein.

### Example 2. Preparation of Three-Dimensional RNA Construct for Direct Reprogramming of Osteoblasts and Confirmation of Effect Thereof

### 2-1. Preparation of Three-Dimensional RNA Construct for Direct Reprogramming of Osteoblasts

A three-dimensional RNA construct expressing octamer-binding transcription factor 4 (OCT4) and core-binding factor subunit beta (CBF-β), which are transcription factors that cause differentiation into osteoblasts, was prepared in the same manner as in Example 1-1. In the OCT4-expressing plasmid and the CBF-β-expressing plasmid, as the ribosome binding site, the Kozak sequence was used instead of the IRES sequence.

The Kozak sequence, OCT4 mRNA encoding sequence, and CBF-β encoding sequence included in the above plasmid, and the unit sequence of the three-dimensional RNA construct expressed from the plasmid containing these are shown in Table 2 below. The unit sequence of the OCT4-expressing RNA construct of SEQ ID NO: 7 includes a T7 promoter, Kozak, and OCT mRNA; the unit sequence of the CBF-β-expressing RNA construct of SEQ ID NO: 8 includes a T7 promoter, Kozak, and CBF-β mRNA. These unit sequences refer to sequences unit in which a promoter, a ribosome binding site, and OCT4 or CBF-β mRNA are operably linked so as to express OCT4, CBF-β, etc.

The prepared three-dimensional RNA constructs were (1) an OCT4-expressing three-dimensional RNA construct in which a transcript including the OCT4 mRNA sequence was self-assembled (hereinafter referred to as O), (2) a CBF-β-expressing three-dimensional RNA construct in which a transcript including the CBF-β mRNA sequence was self-assembled (hereinafter referred to as C), and (3) a three-dimensional RNA construct expressing OCT4 and CBF-β, in which a transcript including the OCT4 mRNA sequence and a transcript including the CBF-β mRNA sequence are mixed in a ratio of 1:1 to 2:1 and self-assembled (hereinafter referred to as OC). In (3), the mixing ratio of the two transcripts can be adjusted depending on the type of cells to be transfected or the ratio of the proteins to be expressed. The RNA constructs (1) to (3) above were prepared such that the average size was 100 nm to 400 nm by adjusting the plasmid concentration.

TABLE 2

| | SEQ | |
|---|---|---|
| Type | ID NO | Sequence |
| Kozak | 4 | GCCACCATGG |
| OCT4 | 5 | ATGGCGGGACACCTGGCTTCGGATTTCGCCTTCTCGCCCCCTC |
| mRNA | | CAGGTGGTGGAGGTGATGGGCCAGGGGGGCCGGAGCCGGGC |
| Encoding | | TGGGTTGATCCTCGGACCTGGCTAAGCTTCCAAGGCCCTCCT |
| Sequence | | GGAGGGCCAGGAATCGGGCCGGGGGTTGGGCCAGGCTCTGA |
| | | GGTGTGGGGGATTCCCCCATGCCCCCCGCCGTATGAGTTCTG |
| | | TGGGGGGATGGCGTACTGTGGGCCCCAGGTTGGAGTGGGGCT |
| | | AGTGCCCCAAGGCGGCTTGGAGACCTCTCAGCCTGAGGGCGA |
| | | AGCAGGAGTCGGGGTGGAGAGCAACTCCGATGGGGCCTCCC |
| | | CGGAGCCCTGCACCGTCACCCCTGGTGCCGTGAAGCTGGAGA |
| | | AGGAGAAGCTGGAGCAAAACCCGGAGGAGTCCCAGGACATC |
| | | AAAGCTCTGCAGAAAGAACTCGAGCAATTTGCCAAGCTCCTG |
| | | AAGCAGAAGAGGATCACCCTGGGATATACACAGGCCGATGT |
| | | GGGGCTCACCCTGGGGGTTCTATTTGGGAAGGTATTCAGCCA |
| | | AACGACCATCTGCCGCTTTGAGGCTCTGCAGCTTAGCTTCAA |
| | | GAACATGTGTAAGCTGCGGCCCTTGCTGCAGAAGTGGGTGGA |
| | | GGAAGCTGACAACAATGAAAATCTTCAGGAGATATGCAAAG |
| | | CAGAAACCCTCGTGCAGGCCCGAAAGAGAAAGCGAACCAGT |
| | | ATCGAGAACCGAGTGAGAGGCAACCTGGAGAATTTGTTCCTG |
| | | CAGTGCCCGAAACCCACACTGCAGCAGATCAGCCACATCGCC |
| | | CAGCAGCTTGGGCTCGAGAAGGATGTGGTCCGAGTGTGGTTC |
| | | TGTAACCGGCGCCAGAAGGGCAAGCGATCAAGCAGCGACTA |
| | | TGCACAACGAGAGGATTTTGAGGCTGCTGGGTCTCCTTTCTC |
| | | AGGGGGACCAGTGTCCTTTCCTCTGGCCCCAGGGCCCCATTT |
| | | TGGTACCCCAGGCTATGGGAGCCCTCACTTCACTGCACTGTA |
| | | CTCCTCGGTCCCTTTCCCTGAGGGGGAAGCCTTTCCCCCTGTC |
| | | TCTGTCACCACTCTGGGCTCTCCCATGCATTCAAACTGA |
| CBF-β | 6 | ATGCCGCGCGTCGTGCCCGACCAGAGAAGCAAGTTCGAGAA |
| mRNA | | CGAGGAGTTTTTTAGGAAGCTGAGCCGCGAGTGTGAGATTAA |
| Encoding | | GTACACGGGCTTCAGGGACCGGCCCCACGAGGAACGCCAGG |
| Sequence | | CACGCTTCCAGAACGCCTGCCGCGACGGCCGCTCGGAAATCG |
| | | CTTTTGTGGCCACAGGAACCAATCTGTCTCTCCAGTTTTTTCC |
| | | GGCCAGCTGGCAGGGAGAACAGCGACAAACACCTAGCCGAG |
| | | AGTATGTCGACTTAGAAAGAGAAGCAGGCAAGGTATATTTG |
| | | AAGGCTCCCATGATTCTGAATGGAGTCTGTGTTATCTGGAAA |
| | | GGCTGGATTGATCTCCAAAGACTGGATGGTATGGGCTGTCTG |
| | | GAGTTTGATGAGGAGCGAGCCCAGCAGGAGGATGCATTAGC |
| | | ACAACAGGCCTTTGAAGAGGCTCGGAGAAGGACACGCGAAT |
| | | TTGAAGATAGAGACAGGTCTCATCGGGAGGAAATGGAGGTG |
| | | AGAGTTTCACAGCTGCTGGCAGTAACTGGCAAGAAGACAAC |
| | | AAGACCCATCAGTTCTGGACCAGCGAGCTGTGCTGCGACTCG |
| | | TGGCGTAATCATGGTCATAGCTGTTTCCTGTGTGAAATTGTTA |
| | | TCCGCTCACAATTCCACACAACATACGAGCCGGAAGCATAAA |
| | | GTGTAA |
| Unit | 7 | UAAUACGACUCACUAUAGG/GAAAUAAGAGAGAAAAGAAGA |
| Sequence | | GUAAGAAGAAAUAUAAGA/GCCACCAUGG/AUGGCGGGACA |
| of RNA | | CCUGGCUUCGGAUUUCGCCUUCUCGCCCCCUCCAGGUGGUG |
| construct | | GAGGUGAUGGGCCAGGGGGGCCGGAGCCGGGCUGGGUUGA |
| Including | | UCCUCGGACCUGGCUAAGCUUCCAAGGCCCUCCUGGAGGGC |
| OCT4 | | CAGGAAUCGGGCCGGGGGUUGGGCCAGGCUCUGAGGUGUG |
| mRNA | | GGGGAUUCCCCCAUGCCCCCCGCCGUAUGAGUUCUGUGGGG |
| | | GGAUGGCGUACUGUGGGCCCCAGGUUGGAGUGGGGCUAGU |
| | | GCCCCAAGGCGGCUUGGAGACCUCUCAGCCUGAGGGCGAAG |
| | | CAGGAGUCGGGGUGGAGAGCAACUCCGAUGGGGCCUCCCCG |
| | | GAGCCCUGCACCGUCACCCCUGGUGCCGUGAAGCUGGAGAA |
| | | GGAGAAGCUGGAGCAAAACCCGGAGGAGUCCCAGGACAUC |
| | | AAAGCUCUGCAGAAAGAACUCGAGCAAUUUGCCAAGCUCC |
| | | UGAAGCAGAAGAGGAUCACCCUGGGAUAUACACAGGCCGA |
| | | UGUGGGGCUCACCCUGGGGGUUCUAUUUGGGAAGGUAUUC |
| | | AGCCAAACGACCAUCUGCCGCUUUGAGGCUCUGCAGCUUAG |
| | | CUUCAAGAACAUGUGUAAGCUGCGGCCCUUGCUGCAGAAG |
| | | UGGGUGGAGGAAGCUGACAACAAUGAAAAUCUUCAGGAGA |
| | | UAUGCAAAGCAGAAACCCUCGUGCAGGCCCGAAAGAGAAA |
| | | GCGAACCAGUAUCGAGAACCGAGUGAGAGGCAACCUGGAG |
| | | AAUUUGUUCCUGCAGUGCCCGAAACCCACACUGCAGCAGAU |
| | | CAGCCACAUCGCCCAGCAGCUUGGGCUCGAGAAGGAUGUGG |
| | | UCCGAGUGUGGUUCUGUAACCGGCGCCAGAAGGGCAAGCG |
| | | AUCAAGCAGCGACUAUGCACAACGAGAGGAUUUUGAGGCU |
| | | GCUGGGUCUCCUUUCUCAGGGGGACCAGUGUCCUUUCCUCU |
| | | GGCCCCAGGGCCCCAUUUUGGUACCCCAGGCUAUGGGAGCC |
| | | CUCACUUCACUGCACUGUACUCCUCGGUCCCUUUCCCUGAG |
| | | GGGGAAGCCUUUCCCCCUGUCUCUGUCACCACUCUGGGCUC |
| | | UCCCAUGCAUUCAAACUGA |
| Unit | 8 | UAAUACGACUCACUAUAGG/GCCACCAUGG/AUGCCGCGCGU |
| Sequence | | CGUGCCCGACCAGAGAAGCAAGUUCGAGAACGAGGAGUUU |
| of RNA | | UUUAGGAAGCUGAGCCGCGAGUGUGAGAUUAAGUACACGG |
| construct | | GCUUCAGGGACCGGCCCCACGAGGAACGCCAGGCACGCUUC |
| Including | | CAGAACGCCUGCCGCGACGGCCGCUCGGAAAUCGCUUUUGU |
| CBF-B | | GGCCACAGGAACCAAUCUGUCUCUCCAGUUUUUUCCGGCCA |
| | | GCUGGCAGGGAGAACAGCGACAAACACCUAGCCGAGAGUA |
| | | UGUCGACUUAGAAAGAGAAGCAGGCAAGGUAUAUUUGAAG |
| | | GCUCCCAUGAUUCUGAAUGGAGUCUGUGUUAUCUGGAAAG |
| | | GCUGGAUUGAUCUCCAAAGACUGGAUGGUAUGGGCUGUCU |
| | | GGAGUUUGAUGAGGAGCGAGCCCAGCAGGAGGAUGCAUUA |
| | | GCACAACAGGCCUUUGAAGAGGCUCGGAGAAGGACACGCG |
| | | AAUUUGAAGAUAGAGACAGGUCUCAUCGGGAGGAAAUGGA |
| | | GGUGAGAGUUUCACAGCUGCUGGCAGUAACUGGCAAGAAG |
| | | ACAACAAGACCCAUCAGUUCUGGACCAGCGAGCUGUGCUGC |
| | | GACUCGUGGCGUAAUCAUGGUCAUAGCUGUUUCCUGUGUG |
| | | AAAUUGUUAUCCGCUCACAAUUCCACACAACAUACGAGCCG |
| | | GAAGCAUAAAGUGUAA |

2-2. Confirmation of Direct Reprogramming from Fibroblasts to Osteoblasts

It was confirmed whether the three-dimensional RNA construct expressing OCT4 and the three-dimensional RNA construct expressing CBF-β could directly reprogram human fibroblasts into osteoblasts.

### (1) Evaluation of Feasibility of Gene Combination

The gene combination feasibility of the three-dimensional RNA construct prepared in Example 2-1 was evaluated. An experimental group (O) was prepared in the same manner as in Example 1, in which human fibroblasts were treated with the OCT4-expressing three-dimensional RNA construct at a concentration of 2 μg/mL and incubated for 24 hours.

Additionally, an experimental group (O+C) was prepared, in which human fibroblasts were treated with an OCT4-expressing three-dimensional RNA construct and a CBF-β-expressing three-dimensional RNA construct at a concentration of 2 μg/mL, respectively, at daily intervals and incubated for 24 hours.

The experimental group (O) and the experimental group (O+C) were incubated in an osteogenic medium containing 10% fetal bovine serum (FBS), β-glycerol phosphate, ascorbic acid, and dexamethasone to induce reprogramming. After 16 days, alkaline phosphatase (ALP), which is an early marker for osteoblast differentiation, was stained and the activity was checked to determine whether fibroblasts were in the stage of conversion to osteoblasts through direct reprogramming.

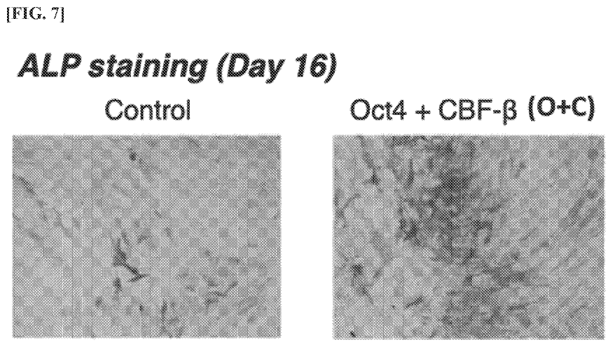

According to FIG. 7, the experimental group (O+C) in which both the OCT4-expressing three-dimensional RNA construct and the CBF-β-expressing three-dimensional RNA construct were transfected, the activity of phosphatase ALP appeared 16 days after the incubation in an osteogenic differentiation medium, thus confirming reprogramming into osteoblasts.

Additionally, the expression level of osteopontin (OPN), which is an osteoblast-specific gene, was quantified through real-time polymerase chain reaction (qRT-PCR) to confirm the suitability of the RNA combination.

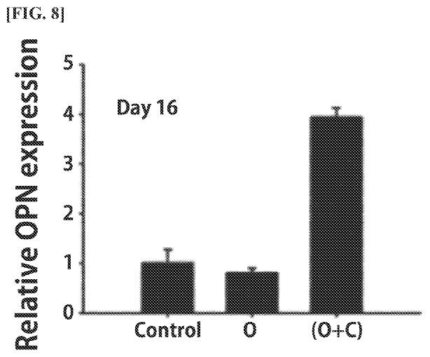

According to FIG. 8, in the experimental group (O) transfected with only OCT4, which is a transcription factor having a role of increasing stemness, there was no significant difference in the expression level of OPN compared to the untreated control group (indicated as Control), and the direct reprogramming into osteoblasts did not occur.

However, in the experimental group (O+C), in which both the OCT4-expressing three-dimensional RNA construct and the CBF-β-expressing three-dimensional RNA construct were transfected, the RNA expression level of OPN increased significantly at 16 days after incubation in an osteogenic differentiation medium. Therefore, it was confirmed that the combination of the OCT4-expressing three-dimensional RNA construct and the CBF-β-expressing three-dimensional RNA construct is suitable for direct reprogramming into osteoblasts.

### (2) Confirmation of Functionality of Directly Reprogrammed Cells

The presence of calcium accumulation in fibroblasts transfected with a three-dimensional RNA construct was confirmed through alizarin red S (ARS) staining to determine whether osteoblast functions were expressed.

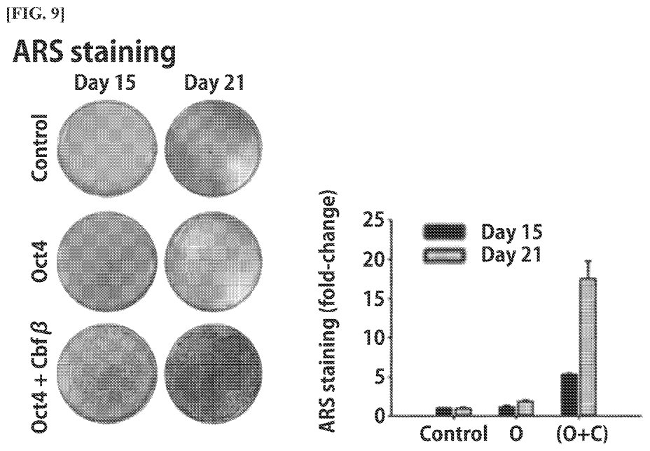

According to FIG. 9, there was almost no increase in ARS staining in the control group (untreated experimental group) and the experimental group (O), in which the OCT4-expressing three-dimensional RNA construct was delivered. However, in the experimental group (O+C), in which the three-dimensional RNA construct for expressing OCT4 and the three-dimensional RNA construct for expressing CBF-β were delivered, the ARS staining increased as the culture period progressed. Therefore, it was confirmed that the fibroblasts transfected with both the OCT4-expressing three-dimensional RNA construct and the CBF-β-expressing three-dimensional RNA construct were directly reprogrammed into osteoblasts and exhibited the function of calcium accumulation.

(3) Confirmation of Function of Three-Dimensional RNA Construct Expressing Both OCT4 mRNA and CBF-β mRNA

Human fibroblasts were treated once with the three-dimensional RNA construct of (3) expressing OCT4 and CBF-β prepared in Example 2-1 at a concentration of 4 μg/mL and incubated for 21 days. In order to confirm the functionality of osteoblasts, the production of mineralized calcium deposits was evaluated through ARS staining.

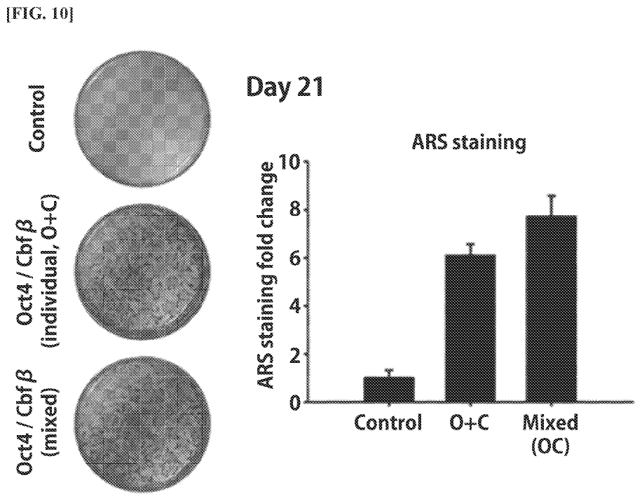

According to FIG. 10, it was confirmed that the ARS staining intensity was higher in the experimental group (mixed, indicated as OC), in which the three-dimensional RNA construct expressing OCT4 and CBF-β was delivered once, compared to the experimental group (individual, indicated as O+C), in which each of the three-dimensional RNA construct expressing OCT4 and the three-dimensional RNA construct expressing CBF-β were delivered at daily intervals. Therefore, it was confirmed that the three-dimensional RNA construct, in which a transcript including OCT4 mRNA and a transcript including CBF-β mRNA were mixed and self-assembled, more efficiently induced direct reprogramming into osteoblasts.

### (4) Confirmation of Direct Reprogramming of Osteoblasts by Osteo-Image Mineralization Analysis

The three-dimensional RNA construct (OC), in which a transcript including OCT4 mRNA and a transcript including CBF-β mRNA are mixed and self-assembled, were transfected into human fibroblasts, and Osteo-Image mineralization analysis was performed on the 7^{th}, 14^{th}, and 21^{st} days to cross-confirm whether the cells possessed functionality as osteoblasts.

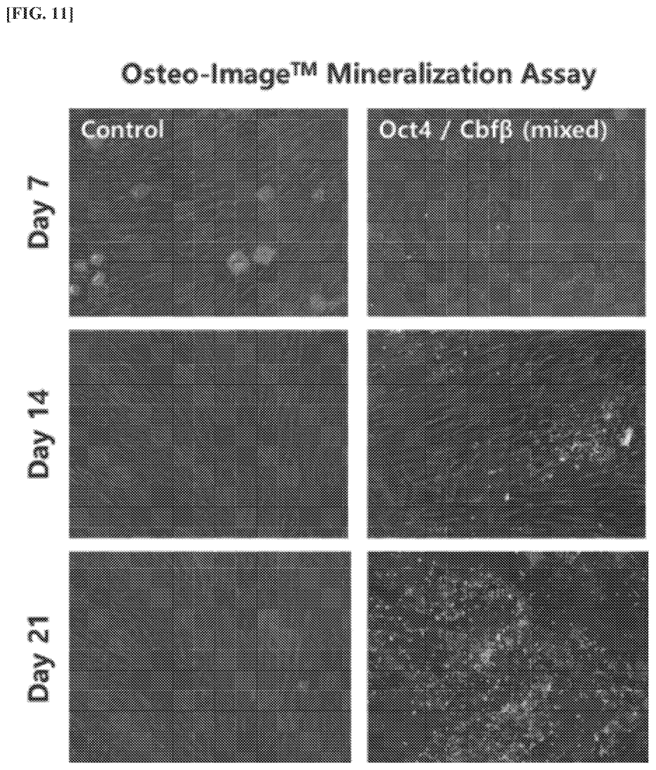

According to FIG. 11, the fibroblasts transfected with the three-dimensional RNA construct (OC), in which a transcript including OCT4 mRNA and a transcript including CBF-β mRNA are mixed and self-assembled, showed an increase in the number of the cells showing green fluorescence (differentiated into osteoblasts) increased significantly over time. Through this, it was confirmed that the human fibroblasts were directly reprogrammed into osteoblasts by the three-dimensional RNA construct, in which a transcript including OCT4 mRNA and a transcript including CBF-β mRNA are mixed and self-assembled.

### (5) Confirmation of Direct Reprogramming of Osteoblasts by Gene Expression Analysis

An experimental group (OC) was prepared, in which osteoblasts were treated at a concentration of 4 μg/mL with a three-dimensional RNA construct, which simultaneously includes OCT4-expressing RNA and CBF-β-expressing RNA, and incubated for 24 hours, and the cells were incubated in an osteogenic medium. The RNA expression levels of OPN, which is a marker of mature osteoblasts, and Runt-related transcription factor 2 (Runx2), which is a marker of early osteoblasts, were analyzed on the 7^{th}, 14^{th}, and 21^{st} days after the incubation by real-time polymerase chain reaction (quantitative RT-PCR).

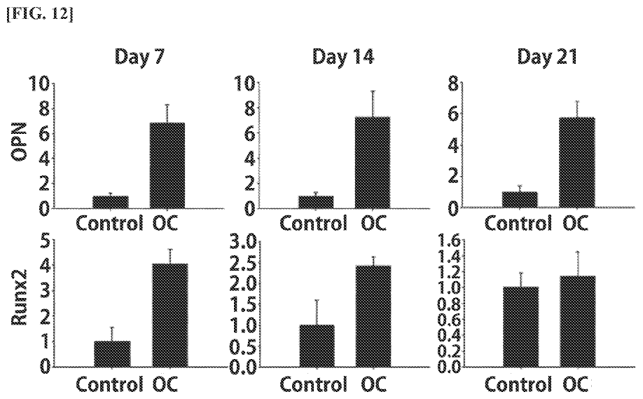

According to FIG. 12, the expression level of Runx2, an early osteoblast marker, was significantly higher in the experimental group (OC) compared to that of the control group on the 7^{th} day, but on the 21^{st} day, the expression in the control group was also increased, and thus the expression level of the experimental group (OC) was similar to that in the experimental group (OC). This is because the cells were cultured in an osteogenic differentiation medium. However, the expression of OPN, a specific marker of mature osteoblasts, was increased only in the experimental group (OC) at all times and the expression was not increased in the control group.

In summary, in the experimental group (OC) where the cells were treated at a concentration of 4 μg/mL with a three-dimensional RNA construct, which simultaneously includes OCT4-expressing RNA and CBF-β-expressing RNA, and incubated for 24 hours, on the 7^{th} day after incubation of the cells in an osteogenic medium, the expressions of both Runx2 (i.e., a marker of early osteoblasts) and OPN (i.e., a marker of mature osteoblasts) are significantly increased and the cells are promptly reprogrammed into osteoblasts and are able to express their functions.

### Example 3. Preparation of Three-Dimensional RNA Construct for Direct Reprogramming of Nerve Cells and Confirmation of Effects Thereof

### 3-1. Preparation of Three-Dimensional RNA Construct for Direct Reprogramming of Nerve Cells

A BRN2-expressing three-dimensional RNA construct was prepared using substantially the same method as Example 1-1 using the BRN2 mRNA-expressing plasmid (including the Kozak sequence) shown in Table 3 below. The average size of the synthesized BRN2-expressing three-dimensional RNA construct was 100 nm to 400 nm.

A three-dimensional RNA construct, which expresses siRNA that inhibits the expression of polypyrimidine tract binding protein (PTB) (i.e., siRNA-PTB), was synthesized through the following two steps.

In the first step, circular DNA for expressing sense RNA for part of the PTB gene and circular DNA for expressing antisense RNA for part of the PTB gene were prepared. Long-stranded DNA (10 μM) of SEQ ID NO: 10 or SEQ ID NO: 11, where the 5′ end is phosphorylated, was mixed with promoter DNA (10 μM) of SEQ ID NO: 12, denaturation was performed at 95° C. for 2 minutes using a thermal cycler, and annealing was performed while gradually lowering the temperature to 25° C. over 1 hour. The resultant was mixed with a reaction solution (30 mM Tris-HCl (pH 7.8), 10 mM MgCl₂, 10 mM DTT, 1 mM ATP) and T4 DNA ligase (3 unit/mL) and T4 DNA ligase (3 units/mL) and the mixture was annealed in a hybridization oven at 25° C. to prepare PTB antisense RNA expressing circular DNA (SEQ ID NOS: 10 and 12) and PTB antisense RNA expressing circular DNA (SEQ ID NOS: 11 and 12).

Since the promoter DNA of SEQ ID NO: 12 was designed to be complementary to 16 bases at the 5′ end and 6 bases at the 3′ end of SEQ ID NOS: 10 and 11 DNA, circular DNA can be prepared by ligation with long-stranded DNA. Additionally, the region corresponding to SEQ ID NO: 12 in circular DNA becomes a binding site and a replication start site (a promoter region) for T7 RNA polymerase.

The second step is to synthesize a three-dimensional RNA construct.

The two types of synthesized circular DNA (PTB sense RNA expression DNA and PTB antisense RNA expression DNA) in an amount of 0.5 μM each, a ribonucleotide solution mix (4 mM), and T7 RNA polymerase (80 units/μL) were mixed with a reaction buffer (80 mM Tris-HCl, 12 mM MgCl₂, 4 mM spermidine, 2 mM dithiothreitol, pH 7.9 (25° C.)) and reacted at 37° C. for 20 hours to synthesize a three-dimensional RNA construct expressing siRNA-PTB.

The three-dimensional RNA construct expressing siRNA-PTB is the one in which an RNA transcript corresponding to part of the PTB sense sequence and an RNA transcript corresponding to part of the PTB antisense sequence are mixed and self-assembled. When the siRNA-PTB expressing three-dimensional RNA construct is transfected into cells, it undergoes continuous Dicer enzyme-mediated cleavage, making it possible to release siRNA including the sequence of SEQ ID NO: 14 or 15, and these siRNAs can bind to the mRNA of PTB thereby playing a role in inhibiting the expression of PTB.

TABLE 3

| | SEQ | |
|---|---|---|
| Type | ID NO | Sequence |
| BRN2 | 9 | ATGGCTACGGCCGCGTCAAACCACTACAGCCTTCTGACTAGT |
| mRNA | | AGTGCCTCTATTGTTCACGCAGAGCCACCGGGTGGAATGCAA |
| Encoding | | CAAGGTGCGGGGGGATACCGCGAAGCCCAGTCACTGGTACA |
| Sequence | | AGGAGACTATGGGGCTTTGCAATCCAACGGTCATCCCCTCAG |
| | | CCATGCCCACCAATGGATTACCGCTCTGAGCCATGGCGGAGG |
| | | AGGTGGAGGTGGAGGGGGAGGCGGAGGTGGCGGAGGTGGG |
| | | GGAGGTGGAGGTGGAGATGGAAGCCCCTGGAGCACGTCCCC |
| | | TCTTGGTCAACCGGACATCAAGCCTAGTGTAGTCGTCCAGCA |
| | | GGGCGGCCGGGGGGATGAACTTCATGGACCCGGAGCCCTTC |
| | | AGCAACAACATCAACAGCAACAGCAACAACAGCAGCAACAG |
| | | CAGCAACAACAACAACAACAGCAACAGCAGCAAAGGCCTCC |
| | | ACACTTGGTTCACCATGCTGCCAATCACCACCCAGGCCCAGG |
| | | CGCATGGAGGTCTGCGGCAGCGGCCGCTCATTTGCCACCTTC |
| | | CATGGGAGCTTCTAACGGCGGTCTTCTGTATTCCCAGCCCTCC |
| | | TTCACGGTAAATGGTATGCTCGGTGCCGGCGGCCAACCGGCC |
| | | GGACTGCATCACCACGGGCTTCGAGATGCCCATGATGAGCCA |
| | | CACCACGCTGATCACCACCCACACCCACATTCACACCCGCAT |
| | | CAGCAGCCTCCTCCTCCGCCACCCCCGCAAGGCCCTCCTGGA |
| | | CACCCGGGTGCCCACCATGATCCACATTCCGACGAGGACACG |
| | | CCTACCTCTGATGATCTGGAACAGTTTGCAAAGCAGTTCAAA |
| | | CAAAGGCGAATAAAGCTCGGCTTTACGCAGGCGGACGTGGG |
| | | GCTCGCGCTGGGCACATTGTATGGGAACGTGTTTTCCCAAAC |
| | | GACCATCTGTCGCTTTGAAGCGTTGCAACTCAGCTTCAAAAA |
| | | TATGTGCAAGTTGAAACCGCTTCTTAATAAGTGGTTGGAGGA |
| | | AGCGGATTCCAGTTCAGGCTCACCAACTAGCATTGACAAAAT |
| | | AGCTGCACAAGGACGCAAAAGGAAGAAGAGAACATCAATAG |
| | | AGGTTTCTGTAAAGGGGGCCTTGGAAAGCCACTTCTTGAAGT |
| | | GTCCTAAACCCTCAGCTCAGGAAATAACATCTCTTGCTGATT |
| | | CTTTGCAGTTGGAAAAGGAGGTAGTGAGGGTCTGGTTTTGCA |
| | | ATCGGCGCCAGAAAGAAAAGAGAATGACGCCACCTGGTGGG |
| | | ACGCTGCCTGGCGCCGAGGACGTCTATGGGGGATCCCGCGAC |
| | | ACACCACCGCACCATGGTGTGCAGACTCCAGTACAATGA |
| Long- | 10 | 5′Phosphate- |
| stranded | | ATAGTGAGTCGTATTAAAGCGTGAAGATCCTGTTCAATATTG |
| DNA1 | | CTGTAGTACAGGTCGACGCAAGCGTGAAGATCCTGTTCAATA |
| (DNA for | | TTATCCCT |
| expressing | | |
| PTB sense | | |
| siRNA) | | |
| Long- | 11 | 5′Phosphate- |
| stranded | | ATAGTGAGTCGTATTAAATATTGAACAGGATCTTCACGCTTC |
| DNA2 | | TAGGCTGGACACACCTCTAAATATTGAACAGGATCTTCACGC |
| (DNA for | | TTATCCCT |
| expressing | | |
| PTB | | |
| antisense | | |
| SIRNA) | | |
| Promoter | 12 | TAATACGACTCACTATAGGGAT |
| DNA | | |
| Unit | 13 | UAAUACGACUCACUAUAGG/GCCACCAUGG/AUGGCUACGGC |
| Sequence of | | CGCGUCAAACCACUACAGCCUUCUGACUAGUAGUGCCUCUA |
| RNA | | UUGUUCACGCAGAGCCACCGGGUGGAAUGCAACAAGGUGC |
| Construct | | GGGGGGAUACCGCGAAGCCCAGUCACUGGUACAAGGAGAC |
| Including | | UAUGGGGCUUUGCAAUCCAACGGUCAUCCCCUCAGCCAUGC |
| BRN2 | | CCACCAAUGGAUUACCGCUCUGAGCCAUGGCGGAGGAGGU |
| mRNA | | GGAGGUGGAGGGGGAGGCGGAGGUGGCGGAGGUGGGGGAG |
| | | GUGGAGGUGGAGAUGGAAGCCCCUGGAGCACGUCCCCUCU |
| | | UGGUCAACCGGACAUCAAGCCUAGUGUAGUCGUCCAGCAG |
| | | GGCGGCCGGGGGGAUGAACUUCAUGGACCCGGAGCCCUUCA |
| | | GCAACAACAUCAACAGCAACAGCAACAACAGCAGCAACAGC |
| | | AGCAACAACAACAACAACAGCAACAGCAGCAAAGGCCUCCA |
| | | CACUUGGUUCACCAUGCUGCCAAUCACCACCCAGGCCCAGG |
| | | CGCAUGGAGGUCUGCGGCAGCGGCCGCUCAUUUGCCACCUU |
| | | CCAUGGGAGCUUCUAACGGCGGUCUUCUGUAUUCCCAGCCC |
| | | UCCUUCACGGUAAAUGGUAUGCUCGGUGCCGGCGGCCAACC |
| | | GGCCGGACUGCAUCACCACGGGCUUCGAGAUGCCCAUGAUG |
| | | AGCCACACCACGCUGAUCACCACCCACACCCACAUUCACAC |
| | | CCGCAUCAGCAGCCUCCUCCUCCGCCACCCCCGCAAGGCCC |
| | | UCCUGGACACCCGGGUGCCCACCAUGAUCCACAUUCCGACG |
| | | AGGACACGCCUACCUCUGAUGAUCUGGAACAGUUUGCAAA |
| | | GCAGUUCAAACAAAGGCGAAUAAAGCUCGGCUUUACGCAG |
| | | GCGGACGUGGGGCUCGCGCUGGGCACAUUGUAUGGGAACG |
| | | UGUUUUCCCAAACGACCAUCUGUCGCUUUGAAGCGUUGCA |
| | | ACUCAGCUUCAAAAAUAUGUGCAAGUUGAAACCGCUUCUU |
| | | AAUAAGUGGUUGGAGGAAGCGGAUUCCAGUUCAGGCUCAC |
| | | CAACUAGCAUUGACAAAAUAGCUGCACAAGGACGCAAAAG |
| | | GAAGAAGAGAACAUCAAUAGAGGUUUCUGUAAAGGGGGCC |
| | | UUGGAAAGCCACUUCUUGAAGUGUCCUAAACCCUCAGCUCA |
| | | GGAAAUAACAUCUCUUGCUGAUUCUUUGCAGUUGGAAAAG |
| | | GAGGUAGUGAGGGUCUGGUUUUGCAAUCGGCGCCAGAAAG |
| | | AAAAGAGAAUGACGCCACCUGGUGGGACGCUGCCUGGCGCC |
| | | GAGGACGUCUAUGGGGGAUCCCGCGACACACCACCGCACCA |
| | | UGGUGUGCAGACUCCAGUACAAUGA |
| RNA | 14 | AGGGAUAAUAUUGAACAGGAUCUUCACGCUUGCGUCGACC |
| Monomer | | UGUACUACAGCAAUAUUGAACAGGAUCUUCACGCUUUAAU |
| Produced | | ACGACUCACUAU |
| from Long- | | |
| stranded | | |
| DNA1 | | |
| C | 15 | AGGGAUAAGCGUGAAGAUCCUGUUCAAUAUUUAGAGGUGU |
| | | GUCCAGCCUAGAAGCGUGAAGAUCCUGUUCAAUAUUUAAU |
| | | ACGACUCACUAU |

3-2. Direct Reprogramming from Fibroblasts to Nerve Cells

It was confirmed whether human fibroblasts could be directly reprogrammed into central nerve cells by delivering the BRN2 expressing three-dimensional RNA construct and the siRNA-PTB expressing three-dimensional RNA construct prepared in Example 3-1 above. The functionality of the converted cells and the ability to regulate gene expression of the three-dimensional RNA construct were confirmed. On the 14^{th} day after delivery of the three-dimensional RNA constructs, the cells converted to central nerve cells were isolated, and on the 28th day, astrocytes, which are auxiliary cells essential for the survival of nerve cells, were added.

### (1) Confirmation of Conversion to Central Nerve Cells Through Direct Reprogramming

After transfecting human fibroblasts with BRN2-expressing three-dimensional RNA construct and siRNA-PTB-expressing three-dimensional RNA construct, the changes in cell phenotype were observed using an optical microscope while incubating for 28 days.

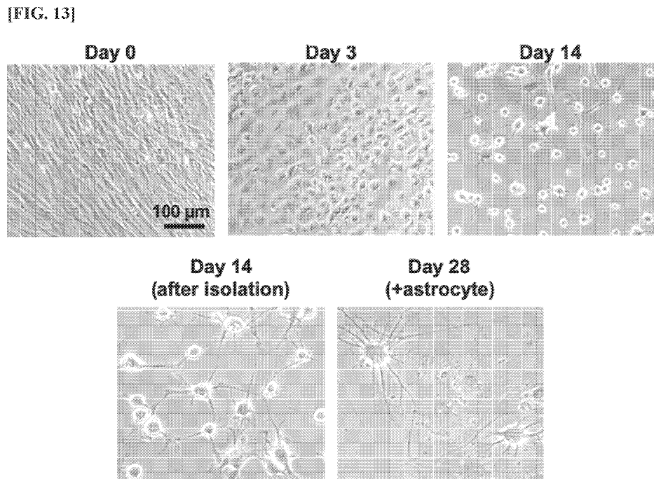

According to FIG. 13, the changes in cell phenotype began on the 3^{rd} day after transfection, and the morphological characteristics of the nerve cells became prominent on the 14^{th} day; therefore, it was confirmed that direct reprogramming to nerve cells was occurring rapidly. In addition, even on the 28^{th} day after transfection, the phenotype of the nerve cells was maintained and the survival rate was high, therefore, it was confirmed that both the BRN2-expressing three-dimensional RNA construct and the siRNA-PTB-expressing three-dimensional RNA construct were sufficiently safe to be maintained for 28 days.

### (2) Confirmation of Functionality of Converted Cells Through Direct Reprogramming

The BRN2-expressing three-dimensional RNA construct and the siRNA-PTB-expressing three-dimensional RNA construct were transfected into human fibroblasts and incubated for 30 days. The reprogrammed cells were immunostained so as to confirm the expression levels of neuron-specific class III-beta tubulin (Tuj1) and microtubule-associated protein 2 (MAP2). Since Tuj1 and MAP2 are nerve cell-specific genes not expressed at all in human fibroblasts, once expressed, they can be acknowledged as exerting nerve cell functions.

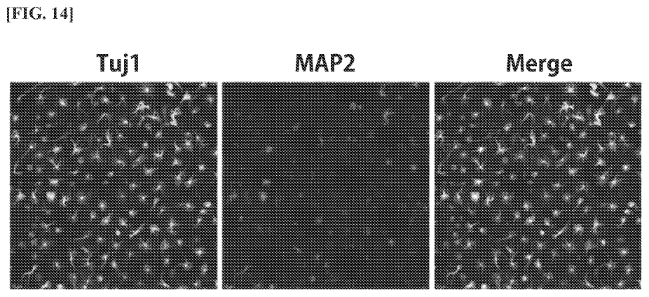

According to FIG. 14, the directly reprogrammed central nerve cells showed high expression levels of Tuj1 and MAP2. Therefore, it was confirmed that when the fibroblasts are transfected with the BRN2-expressing three-dimensional RNA construct and the siRNA-PTB-expressing three-dimensional RNA construct, the cells can be directly reprogrammed into central nerve cells and function as nerve cells.

### (3) Confirmation of Gene Expression Control Ability

The BRN2-expressing three-dimensional RNA construct and the siRNA-PTB-expressing three-dimensional RNA construct were transfected into human fibroblasts and incubated for 30 days. The expression levels of BRN2 and PTB proteins in reprogrammed cells were confirmed using immunocytochemistry. BRN2 is a gene expressed specifically in nerve cells and is not expressed in fibroblasts, whereas PTB is a gene expressed in fibroblasts in large quantities.

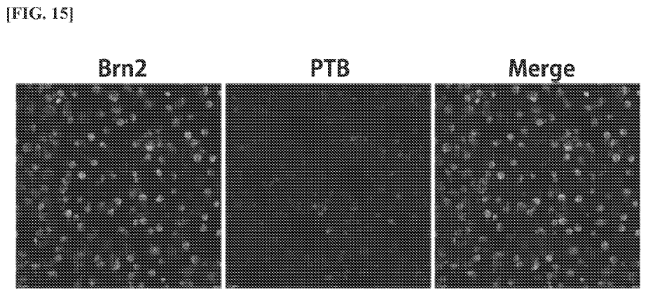

According to FIG. 15, the directly reprogrammed central nerve cells expressed BRN2 at high levels, whereas the expression of PTB was effectively inhibited. Therefore, it was confirmed that the three-dimensional RNA constructs of the present disclosure can effectively regulate the expression of BRN2 and PTB.

Further, according to the experimental results above, a single treatment with the three-dimensional RNA constructs showed an effect of controlling gene expression for 30 days. Considering that the gene expression control effect of existing mRNA introduction lasted only a few hours, the three-dimensional RNA construct of the present disclosure has the advantage in that it shows the effect of controlling a long-term, continuous gene expression with a mere single treatment.

What is claimed is:

1. A composition for inducing direct reprogramming, comprising:
a first active ingredient comprising a three-dimensional RNA construct, in which one or two types of transcripts, each having a repeating unit sequence for the expression of a transcription factor, are mixed and self-assembled;
a second active ingredient comprising a three-dimensional RNA construct, in which one or two types of transcripts, each having a repeating unit sequence for inhibiting the expression of a transcription factor, are mixed and self-assembled; or
a combination thereof.

2. The composition for inducing direct reprogramming of claim 1, wherein the three-dimensional RNA construct has an average size of 100 nm to 400 nm.

3. The composition for inducing direct reprogramming of claim 1, wherein the transcript is transcribed by rolling circle transcription.

4. The composition for inducing direct reprogramming of claim 1, wherein the three-dimensional RNA construct maintains the activity of expressing a transcription factor or the activity of inhibiting the expression of a transcription factor for 30 days or more after transfection into cells.

5. The composition for inducing direct reprogramming of claim 1, wherein the composition for inducing direct reprogramming induces direct reprogramming by one-time transfection into cells.

6. The composition for inducing direct reprogramming of claim 1, wherein the first active ingredient comprises each of a three-dimensional RNA construct, in which the transcripts having a repeating unit sequence for the expression of octamer-binding transcription factor 4 (OCT4) are self-assembled, and a three-dimensional RNA construct, in which the transcripts having a repeating unit sequence for the expression of core-binding factor subunit beta (CBF-β) are self-assembled; or a three-dimensional RNA construct, in which the transcripts having a repeating unit sequence for the expression of OCT4 and the transcript having a repeating unit sequence for the expression of CBF-β are mixed and self-assembled; and
wherein the direct reprogramming is a direct reprogramming from a somatic cell to an osteoblast.

7. The composition for inducing direct reprogramming of claim 6, wherein the unit sequence for OCT4 expression consists of the sequence of SEQ ID NO: 7, and the unit sequence for CBF-β expression consists of the sequence of SEQ ID NO: 8.

8. The composition for inducing direct reprogramming of claim 1, wherein the first active ingredient comprises a three-dimensional RNA construct, in which the transcripts having a repeating unit sequence for the expression of BRN2 are self-assembled; and the second active ingredient comprises a three-dimensional RNA construct, in which the transcripts having a repeating unit sequence for inhibiting the expression of polypyrimidine tract binding protein (PTB) are self-assembled; and
wherein the direct reprogramming is a direct reprogramming from a somatic cell to a nerve cell.

9. The composition for inducing direct reprogramming of claim 8, wherein the unit sequence for BRN2 expression consists of the sequence of SEQ ID NO: 13; the unit sequence for PTB expression consists of the sequence of SEQ ID NO: 14 or SEQ ID NO: 15; and
wherein the three-dimensional RNA construct of the second active ingredient is one in which the transcripts having a repeating unit sequence of SEQ ID NO: 14, the transcripts having a repeating unit sequence of SEQ ID NO: 15, or mixed transcripts thereof are self-assembled.

10. The composition for inducing direct reprogramming of claim 1, wherein the composition for inducing direct reprogramming is for transfection into any one cell selected from the group consisting of fibroblasts, muscle cells, nerve cells, gastric mucosa cells, goblet cells, G cells, pericytes, astrocytes, B cells, blood cells, epithelial cells, neural stem cells, hematopoietic stem cells, mesenchymal stem cells, and umbilical cord blood stem cells.
